(19) United States
(12) Patent Application Publication
(10) Pub. No.: US 2023/0357819 A1
(43) Pub. Date: Nov. 9, 2023

(54) PIPELINE FOR SMALL MOLECULE DISCOVERY

(71) Applicant: Wisconsin Alumni Research Foundation, Madison, WI (US)

(72) Inventors: Jo Handelsman, Verona, WI (US); Marc Chevrette, Madison, WI (US); Chris Thomas, Madison, WI (US); Julia Nepper, Madison, WI (US); Josephine Putnam, Madison, WI (US)

(21) Appl. No.: 18/313,150

(22) Filed: May 5, 2023

Related U.S. Application Data
(60) Provisional application No. 63/338,775, filed on May 5, 2022.

(51) Int. Cl. C12Q 1/18

(52) U.S. Cl. CPC C12Q 1/18

(57) ABSTRACT

The present invention provides streamlined methods for identifying biologically active bacterial products. Products identified by these methods are also provided.

### CROSS-REFERENCE TO RELATED APPLICATIONS

This application claims priority to U.S. Provisional Application No. 63/338,775 filed on May 5, 2022, the contents of which are incorporated by reference in their entireties.

### BACKGROUND

Bacteria are becoming increasingly resistant to our current arsenal of antibiotics (1). As drug resistance spreads globally, we face a growing number of difficult-to-treat infections and deaths. In fact, by the year 2050, antimicrobial-resistant infections are predicted to kill 10 million people annually (2). Further, the COVID-19 pandemic may exacerbate the global burden of antimicrobial resistance, as 72% of patients hospitalized with this viral disease are prescribed antibiotics even though only 7% of these patients have bacterial coinfections (3).

Calls for stepping up the pace of antibiotic discovery are issued regularly (2). Nonetheless, large pharmaceutical companies have largely abandoned this effort, arguing that the antibiotic discovery pipeline is too expensive, time-consuming, and uncertain. Part of their concern is that laborious processes are typically required to determine whether the molecule responsible for the antibiotic activity is new or already known. Thus, there is an unmet need in the art for a pipeline that can be used to efficiently identify novel antibiotics and other biologically active natural products.

### SUMMARY

In a first aspect, the present invention provides methods for identifying biologically active bacterial products. The methods comprise: (a) selecting a bacterium comprising at least one uncharacterized biosynthetic gene cluster; (b) growing the bacterium on a medium in the presence of an induction agent such that the bacterium produces bacterial products; (c) extracting the bacterial products from the medium to form an extract; (d) testing the extract for a biological activity; and (e) identifying the bacterial product that confers the biological activity.

In a second aspect, the present invention provides biologically active bacterial products identified by the methods disclosed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic depiction of methods used to characterize biologically active natural products expressed by selected bacteria. In these methods, the bacteria are grown in petri dishes (i.e., in the presence or absence of antibiotics), the products expressed by the bacteria are extracted from the cultures using an organic solvent (i.e., ethyl acetate), and the resulting crude extracts are subjected to analysis via high-performance liquid chromatography (HPLC).

FIG. 2 shows HPLC ultraviolet-visible spectroscopy (UV-Vis) spectrum contours of extracts (100 µg) from the bacterial isolate TE 3537, which was grown either in the absence of an antibiotic (top panel) or in the presence of rifampicin (rif), tetracycline (tet), or kanamycin (kan).

FIG. 3 shows HPLC UV-Vis spectrum contours of extracts (100 µg) from the bacterial isolate TE 3789, which was grown either in the absence of an antibiotic (top panel) or in the presence of rifampicin (rif), tetracycline (tet), or kanamycin (kan).

FIG. 4 shows HPLC UV-Vis spectrum contours of extracts from the bacterial isolate TE 9994, which was grown either in the absence of an antibiotic (top panel) or in the presence of tetracycline (tet). The dashed white box highlights a peak that represents a product that was induced by tetracycline.

FIG. 5 shows the results of zone of inhibition assays used to screen the TE 9994 extract HPLC fraction that contained the tetracycline-induced product that was detected in FIG. 4. These assays were performed by spotting two amounts of this fraction (i.e., 10 µg and 20 µg) onto plates containing LB (left) and potato dextrose agar (PDA; right) and then applying the test organism B. subtilis. Ampicillin was used as a positive control.

FIG. 6 shows the results of two zone of inhibition assays in which bacterial extract HPLC fractions were screened on two different media. An extract of the bacterial isolate TECH 7, which was grown on PDA in the absence of antibiotic, was subjected to HPLC fractionation. A chromatogram showing the fractionation at a wavelength of 190 nm is shown at the bottom. The resulting fractions were screened for activity against B. subtilis on both PDA (top left) and LB (top right) simultaneously.

### DETAILED DESCRIPTION

The present invention provides streamlined methods for identifying biologically active bacterial products. Products identified by these methods are also provided.

The present inventors have developed a pipeline for identifying biologically active bacterial products. The pipeline utilizes a computational analysis of bacterial genomes to identify bacterial strains that contain novel biosynthetic gene clusters. The identified strains are then grown under induction conditions to induce expression of silent biosynthetic gene clusters. The activities of the products produced by the bacteria are then tested, and the identity of any interesting products is determined.

This pipeline offers two primary advantages. First, it increases the rate of bacterial product discovery by prioritizing interesting products prior to their purification and structural determination. Second, it provides a means to systematically induce expression from silent biosynthetic pathways.

### Methods

In a first aspect, the present invention provides methods for identifying a biologically active bacterial product. The methods comprise: (a) selecting a bacterium comprising at least one uncharacterized biosynthetic gene cluster; (b) growing the bacterium on a medium in the presence of an induction reagent such that the bacterium produces bacterial products; (c) extracting the bacterial products from the medium to form an extract; (d) testing the extract for a biological activity; and (e) identifying the bacterial product that confers the biological activity.

As used herein, a “biologically active bacterial product” is a product produced by a bacterium that has a biological activity. In some embodiments, the biologically active bacterial product is a secondary metabolite. A “secondary metabolite” is a compound that is not required for the growth or reproduction of an organism but is produced to confer a selective advantage to the organism (e.g., increased survivability or fecundity). For example, a secondary metabolite may inhibit the growth of another organism with which the organism competes. Thus, many secondary metabolites inhibit important biological processes.

As used herein, the term “biological activity” refers to an effect that a product has on a living tissue. Examples of biological activities include, without limitation, defensive activities (e.g., antimicrobial activities, pesticide activities, herbicide activities, anticancer activities), catabolic activities, and anabolic activities. However, the present inventors are focused on identifying novel antibiotics. As used herein, an “antibiotic” is a substance that kills or inhibits the growth of a microorganism. Thus, in preferred embodiments, the biological activity is an antimicrobial activity. Suitable antimicrobial activities include, without limitation, antifungal activities, antibacterial activities, antiviral activities, and antiparasitic activities.

In step (a) of the present methods, a bacterium comprising at least one uncharacterized biosynthetic gene cluster is selected. A “biosynthetic gene cluster” is a group of two or more genes that are physically clustered within a particular genome and that all participate in a common biosynthetic pathway (e.g., the synthesis of a secondary metabolite). An “uncharacterized biosynthetic gene cluster” is a biosynthetic gene cluster that is predicted to synthesize a gene product (e.g., based on the presence of one or more open reading frames in the gene cluster), but for which there is no experimental evidence of product synthesis.

In some embodiments, the bacterium is selected based on a genomic analysis. A “genomic analysis” is a method by which genomic features (e.g., DNA sequence, gene structure, gene expression, regulatory elements) are measured or compared on a genomic scale. In some embodiments, the genomic analysis involves using a prioritization algorithm to rank candidate bacteria based on weighted criteria (e.g., criteria related to the novelty and utility of the predicted product of the biosynthetic gene cluster).

Any type of bacterium may be selected in step (a) of the present methods. Suitable bacteria include, without limitation, gram-positive and gram-negative bacteria; spherical shaped (cocci), rod shaped (bacilli), spiral shaped (spirilla), comma shaped (vibrio), and corkscrew shaped (spirochaetes) bacteria; and aerobic and anaerobic bacteria. Soil bacteria are the source of 75% of the antibiotics in current use (7), and there is extraordinary metabolic diversity still to be discovered in soil, which contains on the order of 10⁴ to 10⁶ species of bacteria per gram (4, 5). Thus, in some embodiments, the selected bacterium is from soil.

In step (b) of the present methods, the selected bacterium is grown on a medium in the presence of an induction reagent such that the bacterium produces bacterial products. The bacterium may be grown for any suitable length of time at any suitable temperature. For example, the bacterium may be grown for 1-10 days at a temperature of 4-60° C. In certain embodiments, the bacterium is grown for at 28° C. for four days, as was done in the Examples.

The bacterium may be grown on any suitable medium. As used herein, a “medium” is a liquid or gel designed to support the growth of microorganisms. Examples of standard media that support the growth of a wide variety of bacteria include, without limitation, nutrient agar, tryptic soy agar, brain heart infusion agar, and minimal media. The medium may be enriched by the addition of blood or serum. In the Examples, the inventors grew bacteria on potato dextrose agar because they had previously observed that this medium increases the frequency of antibiotic production compared to other media. Thus, in some embodiments, the medium is potato dextrose agar. Potato dextrose agar is a medium that is made from potato infusion and dextrose. A typical composition of potato dextrose agar comprises about 4 g of potato infusion, about 15-20 g dextrose, and about 20 g agar powder per liter of water. Potato infusion can be made, for example, by boiling 200 grams of sliced unpeeled potatoes in 1 liter of water for 30 minutes and straining the liquid.

Data from whole-genome surveys suggests that many bacterial genes involved in the synthesis of unknown metabolites are not expressed under conventional cultivation conditions. Accordingly, in the present methods, an induction agent is included in the growth medium to help induce the expression of silent genes. As used herein, an “induction agent” is an agent that can be used to promote expression from a biosynthetic gene cluster. Exemplary induction agents include, without limitation, low temperatures, antibiotics, xenobiotics, and coculture with additional bacteria.

Sublethal doses of antibiotics have been shown to induce antibiotic production in bacteria. Thus, in some embodiments, the induction reagent is an antibiotic. As used herein, the term “antibiotic” refers to a reagent that kills or inhibits the growth or reproduction of a bacterium. In the Examples, the inventors grew bacteria on a medium comprising tetracycline, rifampicin, or kanamycin. However, any antibiotic may be used as an induction agent in the present methods. Suitable antibiotics include, without limitation, actinomycin D, ampicillin, carbenicillin, erythromycin, fosmidomycin, gentamicin, kanamycin, neomycin, penicillin, polymyxin B, streptomycin, and vancomycin. In some embodiments, the antibiotic is included in the medium at a sublethal dose. As used herein, a “sublethal dose” is an amount of an antibiotic that is toxic to the bacterium but is not sufficient to kill the bacterium. For example, to achieve sublethal antibiotic doses, the inventors included 2 µg/ml tetracycline, 2 µg/ml rifampicin, or 32 µg/ml kanamycin in their growth medium. Thus, in some embodiments, the antibiotic is tetracycline, and the tetracycline is included in the medium at a concentration of about 2 µg/ml; the antibiotic is rifampicin, and the rifampicin is included in the medium at a concentration of about 2 µg/ml; or the antibiotic is kanamycin, and the kanamycin is included in the medium at a concentration of about 32 µg/ml.

Recent research shows that cocultivation of multiple microbial strains stimulates the production of products (e.g., antibiotics) that are not produced from cultivation of a single strain (6). Thus, in some embodiments, the induction reagent is at least one additional microbe. Microbes (i.e., microorganisms) include bacteria, protozoa, fungi, algae, amoebas, and slime molds. Suitable microbes for use as induction reagents include, without limitation, bacteria and fungi. In some embodiments, the at least one additional microbe is a bacterium is from a different genus than the selected bacterium.

In step (c) of the present methods, the products produced by the bacterium are extracted from the medium to form an extract. The term “extraction” refers to a process of selectively removing a compound of interest from a mixture using a solvent, and the term “extract” refers to a substance obtained via extraction. In some embodiments, the bacterial products are extracted using an organic solvent. An “organic solvent” is a carbon-based substance that can be used to dissolve other substances. Exemplary organic solvents include ethyl acetate, methanol, and acetone. In certain embodiments, the organic solvent is ethyl acetate.

In some embodiments, the methods further comprise fractionating the extract produced in step (c) prior to step (d). “Fractionation” is a separation process in which a mixture is divided into multiple samples based on physical properties (e.g., size, solubility) or chemical properties (e.g., bonding, reactivity). Exemplary fractionation techniques include column chromatography, ion exchange chromatography, size exclusion chromatography, affinity chromatography, and high-performance liquid chromatography (HPLC). In the Examples, the inventors fractionated their extracts using HPLC, which separates molecules based on their differential polarities. Thus, in some embodiments, the extract is fractionated using HPLC.

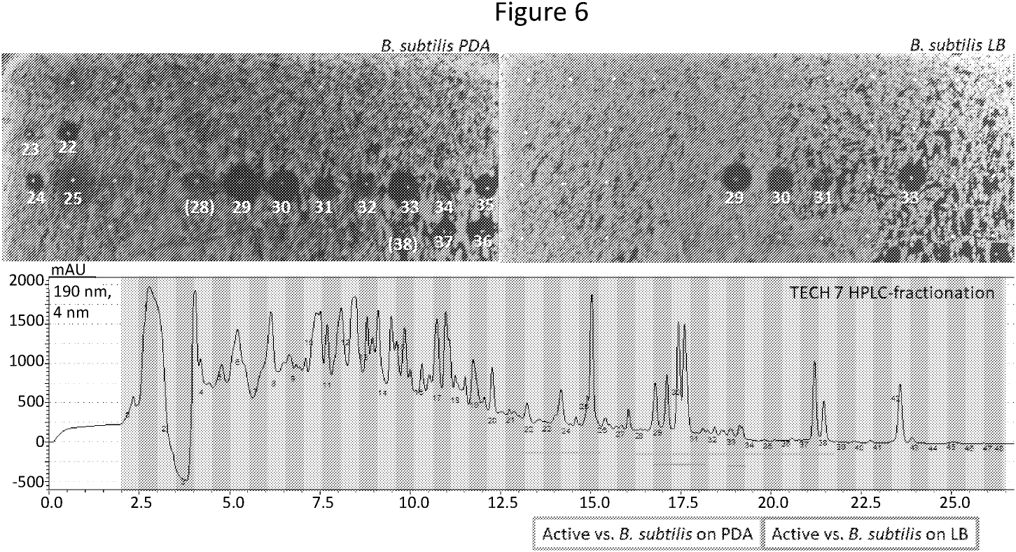

In step (d) of the present methods, the extract is tested for a biological activity. Importantly, the biological activity is characterized in a crude extract before the bacterial product responsible for the activity is identified and purified. The testing method that is utilized will depend on the biological activity of interest. For example, if the biological activity is an enzymatic activity, an enzyme activity assay may be utilized. If the biological activity is an antimicrobial activity, a growth inhibition assay may be utilized. As used herein, a “growth inhibition assay” is method in which the ability of a substance to inhibit the growth of a microbe is tested. Exemplary growth inhibition assays include zone of inhibition assays. For instance, in the Examples, the inventors describe a zone of inhibition assay in which a test organism is grown on a medium that has been spotted or soaked with the bacterial extract and the size of the area in which the extract inhibits the growth of the test organism is measured. The test organism utilized in a growth inhibition assay will depend on the biological activity of interest. For example, a bacterium should be used to test for antibacterial activity whereas a fungus should be used to test for antifungal activity. Suitable test organisms to use to test for the presence of novel antibiotics include safe relatives of ESKAPE pathogens. The ESKAPE pathogens are six nosocomial pathogens that exhibit multidrug resistance and virulence (i.e., Enterococcus faecium, Staphylococcus aureus, Klebsiella pneumoniae, Acinetobacter baumannii, Pseudomonas aeruginosa, and Enterobacter spp). Safe relatives of these notorious pathogens include, without limitation, Bacillus subtilis, Escherichia coli (ATCC 1775), Acinetobacter baylyi (ATCC 33305), Erwinia carotovora, Enterobacter aerogenes (ATCC 51697), and Pseudomonas putida. As is described in the Examples, the inventors found that using potato dextrose agar (PDA) in a zone of inhibition assay stimulates the antimicrobial activity of bacterial products as compared to a standard medium (i.e., LB) (FIG. 6). Thus, in some embodiments the growth inhibition assay is performed on PDA.

In step (e) of the present methods, the bacterial product that confers the biological activity is identified. The product may be identified, for example, using a mass spectrometry (MS)-based method or nuclear magnetic resonance (NMR)-based method. For instance, the inventors utilize solid-phase extraction (SPE) followed by liquid chromatography-mass spectrometry (LCMS) to identify bacterial products. Specifically, to identify bacterial products that were induced by the presence of a particular induction reagent, they compare the LCMS results generated from an extract of a culture that did not comprise the induction reagent to those generated from an extract of a culture that did comprise the induction reagent and look for unique signals in the mass-to-charge data. LCMS data provides the molecular mass of products, which can be translated into a molecular formula and searched for in databases of natural product structures. Alternatively, MS fragmentation data (MS2) can be used for identification (i.e., by searching in spectral databases). These database searches can be conducted using computational methods such as the Compound Discoverer™ software (Thermo Fisher).

As used herein, the term “identifying” does not require that the bioactive bacterial product has been purified or that its exact chemical structure has been obtained. Instead, this term may merely indicate that one has determined that a particular biologically active product can be synthesized by a particular bacterium under a particular set of conditions. The biologically active bacterial product can thus be identified in either a crude extract or a sample that has undergone further processing (e.g., fractionation).

### Bacterial Products

In a second aspect, the present invention provides biologically active bacterial products identified by the methods disclosed herein. The biologically active bacterial products may be useful in a wide variety of applications including as medicines, agrochemicals, flavoring agents, nutrients, or pigments.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument, and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1

The present invention provides a pipeline for identifying novel, biologically active natural products expressed by bacteria. In the following example, the inventors describe methods they use to characterize the products expressed by the bacteria that are selected using this pipeline. In these methods, the bacteria are grown in petri dishes (i.e., in the presence or absence of antibiotics), the products expressed by the bacteria are extracted from the cultures using an organic solvent, and the extracts are subjected to analysis via high-performance liquid chromatography (HPLC), zone of inhibition assays, and solid-phase extraction (SPE) followed by liquid chromatography-mass spectrometry (LCMS). In some cases, the extracts are fractionated via HPLC, and the resulting fractions are subjected to analysis.

### Materials and Methods

### Culture Conditions and Extract Production

Overnight cultures of bacterial isolates were grown in lysogeny broth (LB; BD-Difco LB, 1-5 mL) at 28° C. with shaking at 200 rpm. Turbid cultures (100 µL) were spread onto 100 mm style petri dishes of potato dextrose agar (PDA; BD-Difco Potato Dextrose Broth with 15 g/L bacteriological grade agar), both with and without a sublethal concentration of an antibiotic (i.e., tetracycline, rifampicin, or kanamycin) included in the agar. Cultures were incubated at 28° C. for four days. For extraction, agar cakes were individually excised and placed in a 50 mL conical tube, chopped into small pieces within the tube, and suspended in 15 mL of ethyl acetate such that it completely covered the agar. Agar was extracted for at least one hour on a benchtop platform shaker that was shaking at 300 rpm at room temperature. The tubes were subsequently centrifuged (4000 rpm, 5 minutes) and then decanted into 20 mL sample vials. The decanted extract was dried in a Thermo Scientific™ Savant SpeedVac™ vacuum concentrator (40° C., volatile setting) for about 1.5 hours. The extractions were repeated two more times (for a total of three times), and the extracts were combined into one vial per sample each time. The resulting crude extracts were then dried overnight (35° C., volatile setting) and stored at -20° C. Extracts were resuspended in 700 µL of HPLC grade methanol (MeOH) and filtered (0.22 µm, PVDF) into pre-weighed 2 mL HPLC vials. Samples were dried, massed, and stored at -20° C. until they were used for analysis.

### Extract Preparation for Bioactivity Screening

Extracts were dissolved at 10 mg/mL in HPLC grade MeOH, transferred (30 µL, 300 µg) to a 300 µL 96-shallow conical well plate, and dried down in the SpeedVac™. Dried extracts were resuspended in 30 µL DMSO. Extracts were stamped onto agar omni trays (1-3 µL per spot) for zone of inhibition assays.

### Extract Preparation for HPLC Screening

Extracts were dissolved at 10 mg/mL in HPLC grade MeOH. Then, 10 µL of each dissolved extract was diluted into 40 µL of MeOH in 300 µL polypropylene autosampler vials, and the vials were set in the autosampler of a Shimadzu Nexera HPLC. Samples were injected (48 µL) onto an analytical Phenomenex C-18(2) Luna HPLC column (250 × 4.6 mm, 5 µm particle size) and separated using a gradient of acetonitrile (ACN; line B) and Milli-Q® water (MQ H₂O; line A), each containing 0.1% acetic acid or 0.05% tri-fluoro acetic acid (TFA), with a flow rate of 1 mL/min. The method protocol was as follows: 10% B for 0.5 min, linear ramp to 100% B until 15 min, hold at 100% B until 20 min, linear ramp to 10% B until 20.5 min, equilibrate at 10% B until 27 min. Effluent compounds were detected using a photo diode array detector (scan range: 190 - 600 nm) using a D₂ lamp. Select wavelength chromatograms and ultraviolet-visible spectroscopy (UV-Vis) spectrum contours were used to compare sample profiles.

### HPLC Fractionation

Extracts were dissolved in HPLC grade MeOH and injected (0.5 mg × 3, 1.5 mg total) onto an analytical Phenomenex C-18(2) Luna HPLC column (250 × 4.6 mm, 5 µm particle size) using the same conditions that were used for the HPLC analysis of extracts. Effluent was collected in a 2 mL 96-deep well plate at approximately 30-second intervals, beginning at 2 minutes and ending at 26 minutes, resulting in 48 fractions. Well plates were dried down in a SpeedVac™ (35° C.). The fraction in each well was resuspended in 200 µL MeOH, dispensed into a 300 µL 96-shallow conical well plate, dried down in the SpeedVac™, and resuspended in 30 µL DMSO. Individual fractions were spotted onto agar omni-trays (1-3 µL per spot) for zone of inhibition assays.

### Zone of Inhibition Assays

Test organisms were cultured overnight in LB medium (28° C., shaking 200 rpm) one day prior to analysis. Activity against test organisms was assayed on various media, including Luria-Bertani (LB) and potato dextrose agar (PDA). The overnight growths of the test organisms were diluted (dependent on organism and testing medium) and then spread using sterile glass beads on agar plates that had been spotted or soaked with crude extract or extract fractions. Ampicillin and polymyxin B were used as bioactive positive control compounds for gram-positive and gram-negative test organisms, respectively. The plates were incubated at 28° C. overnight and analyzed the following day for zones of inhibition, both by eye and using a plate imager.

### SPE and LCMS

Extracts were dissolved in HPLC grade MeOH at 10 mg/mL. 1 µL (10 ug) of the dissolved extracts was diluted into 500 µL of a 10% MeOH solution in MQ H₂O in a 96-well plate and subjected to SPE over a C-18 cartridge (Thermo Scientific Hypersep, 50 mg) using a Gilson GX-271 liquid handler. This step was conducted to minimize lipid deposits onto the LCMS column, which would otherwise negatively impact analyses. The protocol was as follows: equilibrate with 990 µL 95% MeOH, load 990 µL 5% MeOH, load 450 µL sample, wash with 990 µL 5% MeOH, and elute with 200 µL 95% MeOH. Samples were eluted into 200 µL LCMS vials with glass inserts.

LCMS was conducted using a Q-Exactive™ Plus Orbitrap Mass Spectrometer (Thermo Fisher) coupled with a Vanquish™ UHPLC system (Thermo Scientific). Samples were injected (5µL) onto a Phenomenex Kinetex C-18 column (100 × 2.6 mm, 2.1 µm) using a 20-minute gradient of ACN (line B) and H₂O (line A), each containing 0.1% formic acid, with a constant flow rate of 0.3 mL/min. The protocol was as follows, beginning at 10% B: linear ramp to 40% until 9 min, linear ramp up to 97% B until 13 min, hold at 97% B until 16 min, linear ramp to 10% B until 17 min, and equilibrate at 10% until 20 min.

Mass spectra data was collected for 17 minutes and then the sample was diverted to waste during the final equilibration. Data-dependent acquisition was used in positive mode, selecting the top five ions for MS-MS analysis. Normalized collision energy (NCE) was ramped between 20-50-100 for ion fragmentation. MS1 scans were collected at 70 k resolution, and MS2 scans were collected at 17.5k.

### LCMS Data Analysis

LCMS data was analyzed using Compound Discoverer™ 3.3 Software (Thermo Fisher). The workflow utilized was modified from one of the “Metabolomics” workflow presets. The Thermo mzCloud database (MS2), the Bamba lab polar metabolites spectral library (provided by Thermo, MS2), and the NP Atlas, BioCyc, Kegg, Human Metabolome structure databases from ChemSpider (MS1) were searched to identify compounds. Thermo mzLogic was applied for ChemSpider searches. Molecular formulas were also predicted for the compounds. In general, data from bacteria grown under different induction conditions were compared using a suite of tools offered by Compound Discoverer, including p-value assessments, box-and-whisker plots of specific compounds, and molecular networks of compounds based on MS2 data. Compounds that were unique to a particular condition were flagged for further analysis.

### Results

### Growing Bacteria in the Presence of Antibiotics Alters Metabolite Production

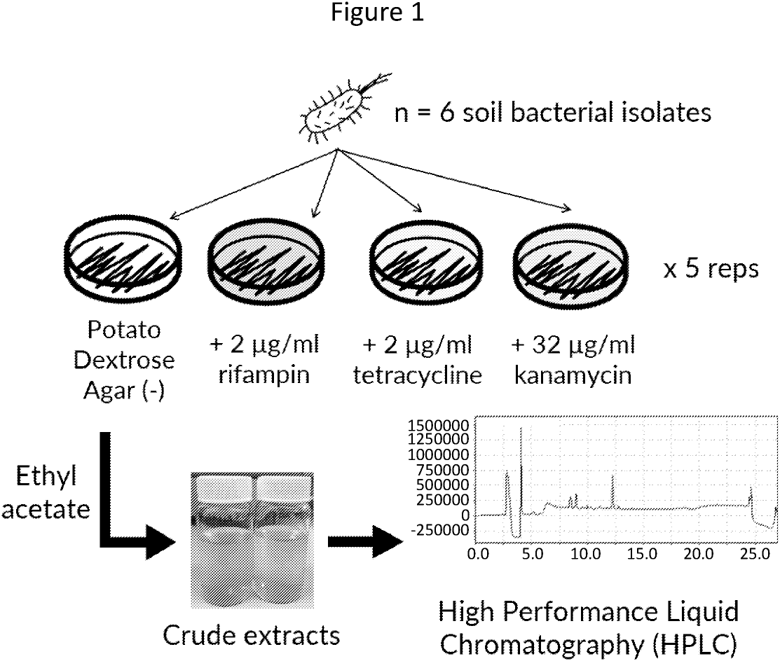

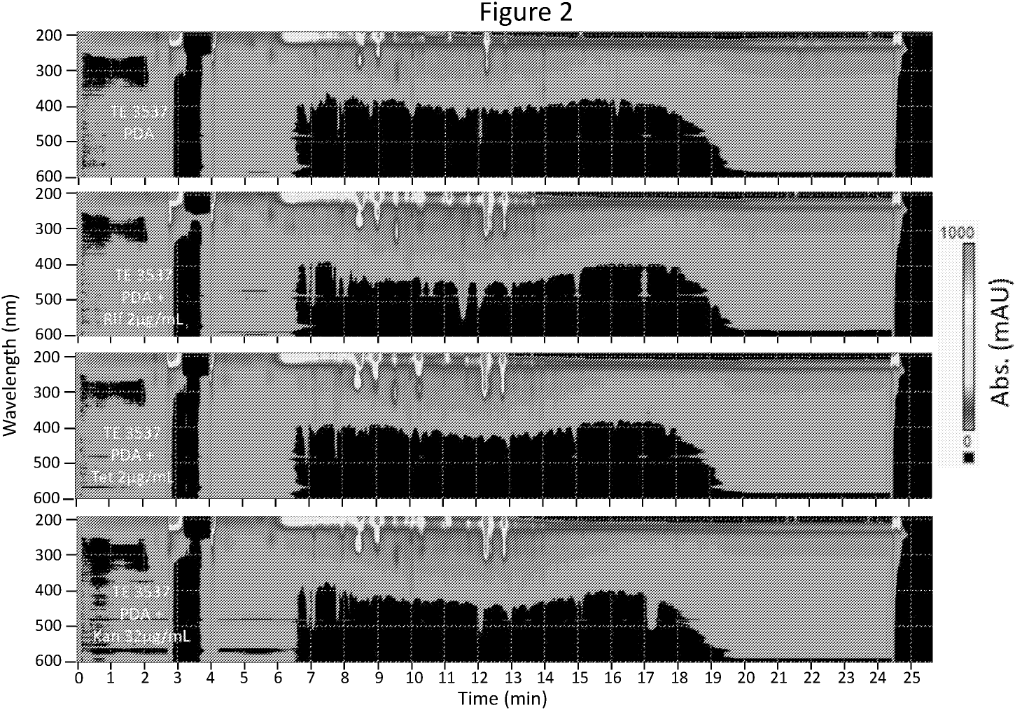

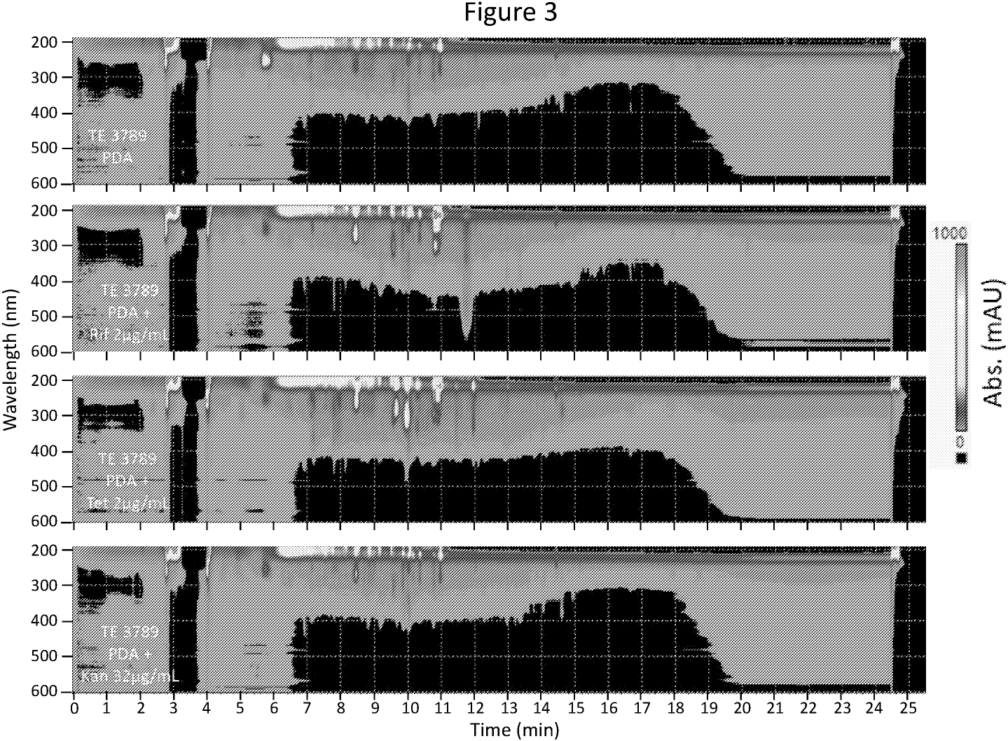

Sublethal doses of antibiotics are known to induce antibiotic production. Thus, to attempt to induce expression of novel biologically active bacterial products, we grew two bacterial isolates (i.e., TE 3537 and TE 3789) on potato dextrose agar with or without one of three antibiotics (i.e., kanamycin, tetracycline, and rifampicin). Products were extracted from the bacteria using ethyl acetate, and the crude extracts were subjected to HPLC. See FIG. 1 for a schematic depiction of this workflow. In all isolates tested, the presence of the antibiotics in the growth medium altered the profile of products produced by the bacteria. In some cases, the changes were universal (i.e., similar across all three antibiotics), while in others they were antibiotic specific. For example, the metabolite profile of the bacterial isolate TE 3537 did not change substantially when this bacterium was grown in the presence of the antibiotics kanamycin, tetracycline, and rifampicin (FIG. 2), though there were some subtle differences between certain conditions. Namely, the profiles generated in the antibiotic-induced conditions between the 12- and 14-minute retention times are generally the same but are distinct from the profile generated in the no-antibiotic control condition. Subtle shifts in the compounds produced are also observable in some instances, e.g., at the 10.25-minute retention time for the tetracycline condition and at the 11.15-minute retention time for the rifampicin condition. In contrast, the metabolite profile of the bacterial isolate TE 3789 differed in every condition (FIG. 3). Kanamycin did not elicit any strong changes as compared to the no-antibiotic control, but rifampicin and tetracycline each elicited a unique metabolite profile.

### Fractionation can be Used to Enrich for Antibiotic-Induced Products

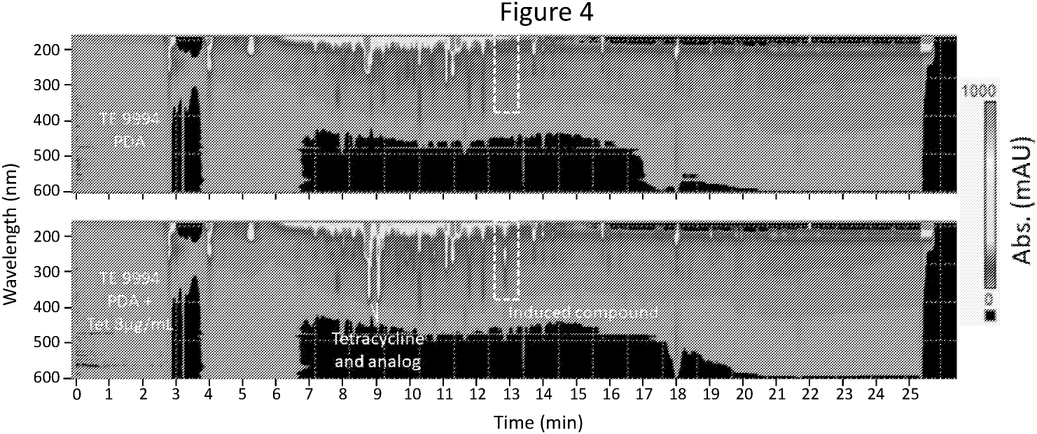

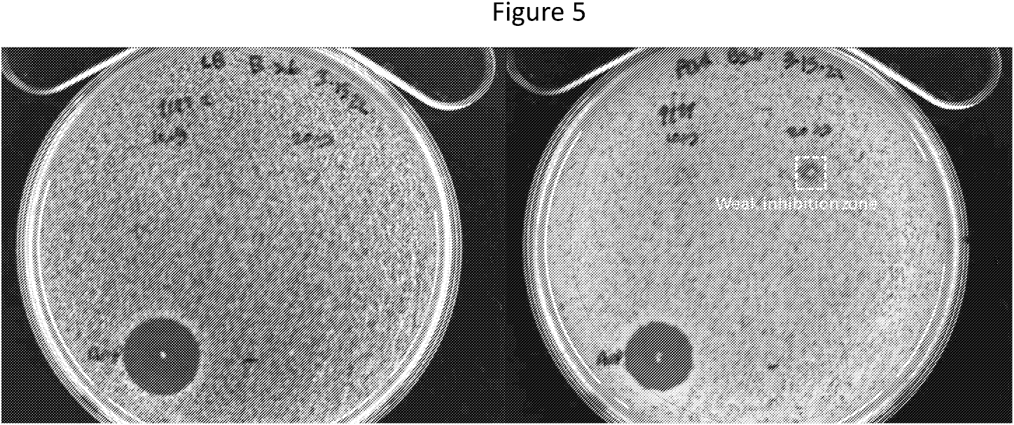

By fractionating the bacterial extracts via HPLC, we can home in on specific products that were induced by the presence of an antibiotic in the growth medium. For example, an HPLC analysis of extracts from the bacterial isolate TE 9994 grown in the presence or absence of tetracycline showed that tetracycline induces the expression of a product that has a retention time between 12 and 13 minutes (FIG. 4). Thus, we used HPLC to fractionate the crude extract from this bacterium and selected the fraction containing the induced product for further analysis. When this fraction was tested in a zone of inhibition assay, it inhibited the growth of B. subtilis on PDA but not on LB (FIG. 5).

### PDA Stimulates Antimicrobial Activity

To determine whether the medium used in a zone of inhibition assay influences the assay results, we compared the results of two assays performed on different media. The extract we used in these assays was HPLC-fractionated extract from the bacterial isolate TECH 7, which was grown on PDA in the absence of antibiotics. When the TECH 7 extract fractions were screened for activity against B. subtilis on PDA and LB media simultaneously, a larger proportion of the fractions inhibited the growth of B. subtilis on PDA than on LB (FIG. 6). Thus, PDA appears to stimulate the antimicrobial activity of products produced by this bacterium. This activity would have been missed if the fractions were screened on LB alone.

### REFERENCES

1. Centers for Disease Control. 2019. Antibiotic resistance threats in the United States, 2019. U.S. Department of Health and Human Services, Centers for Disease Control, Atlanta, GA. doi:10.15620/cdc:82532.

2. Review on Antimicrobial Resistance. 2016. Tackling drug-resistant infections globally: final report and recommendations. amr-review.org/sites/default/files/160518_Final%20paper_with%20cover.pdf.

3. Langford BJ, So M, Raybardhan S, Leung V, Westwood D, MacFadden DR, Soucy J-PR, Daneman N. 2020. Bacterial co-infection and secondary infection in patients with COVID-19: a living rapid review and meta-analysis. Clin Microbiol Infect 26:1622-1629.

4. Schloss PD, Handelsman J. 2006. Toward a census of bacteria in soil. PLoS Comp Biol 2:e92.

5. Dykhuizen D. 2005. Species numbers in bacteria. Proc Calif AcadSci 56:62-71.

6. Ueda K, Beppu T. 2017. Antibiotics in microbial coculture. J Antibiot (Tokyo) 70:361-365. doi:10.1038/ja.2016.127.

7. Bérdy J. 2005. Bioactive microbial metabolites. J Antibiot (Tokyo) 58:1-26.

What is claimed:

1. A method for identifying an antimicrobial bacterial product, the method comprising:
a) selecting a bacterium comprising at least one uncharacterized biosynthetic gene cluster;
b) growing the bacterium on a medium in the presence of an induction reagent such that the bacterium produces bacterial products;
c) extracting the bacterial products from the medium to form an extract;
d) testing the extract for an antimicrobial activity;
e) identifying the bacterial product that confers the antimicrobial activity.

2. The method of claim 1, wherein the bacterium is selected via a genomic analysis in step (a).

3. The method of claim 1, wherein the bacterium selected in step (a) is from soil.

4. The method of claim 1, wherein the bacterium is grown for at 28° C. for four days in step (b).

5. The method of claim 1, wherein the medium used in step (b) is potato dextrose agar.

6. The method of claim 1, wherein the induction reagent used in step (b) is an antibiotic.

7. The method of claim 6, wherein the antibiotic is tetracycline, rifampicin, or kanamycin.

8. The method of claim 7, wherein the antibiotic is included in the medium at a sublethal dose.

9. The method of claim 8, wherein:
a) the antibiotic is tetracycline, and the tetracycline is included in the medium at a concentration of about 2 µg/ml;
b) the antibiotic is rifampicin, and the rifampicin is included in the medium at a concentration of about 2 µg/ml; or
c) the antibiotic is kanamycin, and the kanamycin is included in the medium at a concentration of about 32 µg/ml.

10. The method of claim 1, wherein the induction reagent is at least one additional microbe.

11. The method of claim 10, wherein the at least one additional microbe includes a bacterium from a different genus.

12. The method of claim 1, wherein the bacterial products are extracted using an organic solvent in step (c).

13. The method of claim 12, wherein the organic solvent is ethyl acetate.

14. The method of claim 1, wherein the method further comprises fractionating the extract produced in step (c) prior to step (d).

15. The method of claim 14, wherein the extract is fractionated using high-performance liquid chromatography (HPLC).

16. The method of claim 1, wherein the antimicrobial activity of the extract is tested using a growth inhibition assay in step (d).

17. The method of claim 16, wherein the growth inhibition assay is performed on potato dextrose agar.

18. The method of claim 1, wherein the bacterial product that confers the antimicrobial activity is identified using a mass spectrometry-based method in step (e).

19. The method of claim 18, wherein the mass spectrometry-based method is solid-phase extraction (SPE) followed by liquid chromatography-mass spectrometry (LCMS).

20. An antimicrobial bacterial product identified by the method of claim 1.
